(19) United States
(12) Patent Application Publication
(10) Pub. No.: US 2026/0174547 A1
(43) Pub. Date: Jun. 25, 2026

(54) INTRAOCULAR LENS DEVICES HAVING PERIPHERAL BENDABLE MEMBRANE SEGMENTS FOR OPTICAL MATERIAL DISPLACEMENT AND METHODS OF USE

(71) Applicant: ForSight Vision6, Inc., South San Francisco, CA (US)

(72) Inventors: Nicole Kahn-Dror, South San Francisco, CA (US); Guy Oren, South San Francisco, CA (US); Matthew Clarke, South San Francisco, CA (US); Nadav Peleg, South San Francisco, CA (US)

(21) Appl. No.: 19/124,770

(22) PCT Filed: Oct. 30, 2023
(86) PCT No.: PCT/US2023/036284

§ 371 Date: Apr. 25, 2025

Related U.S. Application Data
(60) Provisional application No. 63/420,874, filed on Oct. 31, 2022.

(51) Int. Cl. A61F 2/16; A61F 9/00

(52) U.S. Cl. CPC A61F 2/16; A61F 2/1613; A61F 2/1629; A61F 2/1635; A61F 2/1648; A61F 9/0017; A61F 2002/1681; A61F 2002/1682; A61F 2250/0018

(57) ABSTRACT

A lens device for treatment of an eye having a lens capsule with an internal chamber bound, in part, by a dynamic anterior optic and having a perimeter region. A deformable region having a three-dimensional bellows shape defining a volume projects radially outward from the perimeter region of the lens capsule and has an outer membrane segment configured to bend around a hinge relative to an inner membrane segment. A volume of an optical liquid is contained within the internal chamber and the volume of the deformable region. A force translation arm extends radially outward from the deformable region. Upon implantation of the lens device, the force translation arm directly contacts ciliary tissue to harness ciliary body movements that compresses and reduces the volume of the deformable region displacing optical liquid contained within the volume of the deformable region towards the internal chamber.

### CROSS-REFERENCE TO RELATED APPLICATIONS

The present application claims the benefit of priority to co-pending U.S. Provisional Application Ser. No. 63/420,874, filed Oct. 31, 2022, the entire contents of which are hereby incorporated by reference herein in its entirety.

### BACKGROUND

IOLs are typically implanted after cataract extractions. Generally, IOLs are made of a foldable material, such as silicone or acrylics, for minimizing the incision size and improving patient recovery time. Most commonly used IOLs are single-element lenses that provide a single focal distance for distance vision. Accommodating intraocular lenses (AIOLs) have also been developed to provide adjustable focal distances (or accommodations) that rely on the natural focusing ability of the eye, for example, as described in U.S. Pat. Nos. 8,414,646, 8,167,941, 9,913,712, 10,258,805, and US 2019/0269500, which are each incorporated by reference herein in their entireties.

IOLs are beneficial for patients not suffering from cataracts, but who wish to reduce their dependency on glasses and contacts to correct their myopia, hyperopia and presbyopia. Intraocular lenses used to correct large errors in myopic, hyperopic, and astigmatic eye are called “phakic intraocular lenses” and are implanted without removing the crystalline lens. In some cases, aphakic IOLs (not phakic IOLs) are implanted via lens extraction and replacement surgery even if no cataract exists. During this surgery, the crystalline lens is extracted and an IOL replaces it in a process that is very similar to cataract surgery. Refractive lens exchange, like cataract surgery, involves lens replacement, requires making a small incision in the eye for lens insertion, use of local anesthesia and lasts approximately 30 minutes.

IOLs, particularly AIOLs, may incorporate liquids in fluid chambers such that accommodation is achieved with the help of fluid-actuated mechanisms. A force exerted on a portion of the lens is transmitted via the fluid to deform a flexible layer of the lens resulting in accommodative shape change of the IOL. For example, ciliary muscle movements of the eye may be harnessed by components of an AIOL to drive shape change and accommodation. The AIOLs can achieve an optical power or diopter (D) in a desired range due to shape change of the optic upon application of a small amount of force (e.g., as little as 0.1-1.0 grams force (gf)) applied by the eye tissue. The AIOLs provide reliable dioptric change by harnessing small forces. A chamber for containing liquid materials that is formed by flexible layers of elastomeric material can change shape and thus, power of the lens depending on the volume of liquid. As fill volume increases beyond the chamber volume, the flexible layers can bulge outward creating a lens with a greater focal length.

There is need in the art for improved manufacturing of lenses that provide improved properties for patients in need. The disclosure is directed to this, as well as other, important ends.

### SUMMARY

In an aspect, provided is a lens device for treatment of an eye including a lens capsule having an internal chamber bound, in part, by a dynamic anterior optic and having a perimeter region. The lens device has a deformable region having a three-dimensional bellows shape defining a volume. The deformable region is projecting radially outward from the perimeter region of the lens capsule. The deformable region includes an outer membrane segment configured to bend around a hinge relative to an inner membrane segment. The lens device has a volume of an optical liquid contained within the internal chamber and the volume of the deformable region. The lens device has a force translation arm extending radially outward from the deformable region. Upon implantation of the lens device in the eye, the force translation arm directly contacts ciliary tissue to harness ciliary body movements that compresses and reduces the volume of the deformable region displacing optical liquid contained within the volume of the deformable region towards the internal chamber.

The bellows can be curved or angular. The force translation arm can extend radially outward and posteriorly at an angle relative to a plane of the dynamic anterior optic. The angle can be about 5 to about 10 degrees. The angle can match an angle of ciliary body movement in anterior direction relative to the lens.

In an interrelated aspect, provided is a lens device for treatment of an eye including a lens capsule having an internal chamber bound, in part, by a dynamic anterior optic. The lens device has a deformable region having a three-dimensional curved bellows shape defining a volume. The deformable region projects radially outward from a perimeter region of the lens capsule. The deformable region includes an outer membrane segment configured to move relative to an inner membrane segment around a hinge linking the outer membrane segment to the inner membrane segment. The lens device has an optical liquid present within the internal chamber and the volume of the deformable region; and a force translation arm extending radially outward from the deformable region. Upon implantation of the lens device in the eye, the force translation arm directly contacts ciliary tissue to harness ciliary body movements that compresses and reduces the volume of the deformable region displacing optical liquid contained within the volume of the deformable region towards the internal chamber.

The outer membrane segment can include an anterior-facing surface and a posterior-facing surface. The deformable region has an outside height (A) between the anterior-facing surface of the outer membrane segment and the posterior-facing surface of the outer membrane segment. The outside height (A) of the deformable region can be about 0.4 mm-0.7 mm. The inner membrane segment can include an inner surface on an anterior region and an inner surface on a posterior region of the inner membrane segment. The deformable region has an inside height (E) between the inner surface on the anterior region of the inner membrane segment and the inner surface on the posterior region of the inner membrane segment. The inside height (E) of the deformable region can be about 0.4 mm-0.8 mm.

Inner surfaces of the hinge can create a narrowing in the volume of the deformable region separating an outer portion of the volume from an inner portion of the volume. The narrowing in the volume can form a nozzle segment within the deformable region. The nozzle segment has a height (B) between an inner surface of the hinge on an anterior side and an inner surface of the hinge on a posterior side. The height (B) of the nozzle segment can be about 0.15 mm-0.4 mm. The inner membrane segment can include an external surface facing generally radially outward and the outer membrane segment can include an external surface facing generally radially inward. There can be a distance between the external surface of the inner membrane segment and the external surface of the outer membrane segment and an angle between the external surface of the inner membrane segment and the external surface of the outer membrane segment. The distance can be about 0.2 mm-0.6 mm. The distance and the angle can be configured to change upon application of a force causing movement of the outer membrane segment relative to the inner membrane segment. A curvature of the inner membrane segment relative to the lens capsule can flatten. The deformable region can have an inside height (E) of about 0.60 mm-0.65 mm and provide a displaced volume of optical liquid that can be about 0.2 mm³-0.5 mm³ at about 1.3-1.7 grams applied force. The displaced volume of optical liquid can be about 0.3 mm³. The deformable region can have an inside height (E) of about 0.45 mm-0.55 mm and provide a displaced volume of optical liquid that is about 0.15 mm³-0.35 mm³ at about 1.3-2.0 grams applied force. The displaced volume of optical liquid can be about 0.2 mm³.

An anterior region of the outer membrane segment can compress a first distance upon application of a first force to the anterior region and a posterior region of the outer membrane segment can compress a second distance upon application of a second force. The first distance and the second distance can be the same and the first force and the second force can be different. The force translation arm can include a sloped external surface where direct contact between the force translation arm and ciliary tissue occurs during ciliary body movements.

In an interrelated aspect, provided is a lens device for treatment of an eye including a lens capsule having an internal chamber bound, in part, by a dynamic anterior optic, the lens capsule having a perimeter region. The lens device includes a deformable region having a three-dimensional angular bellows shape defining a volume. The deformable region projects radially outward from the perimeter region of the lens capsule. The deformable region has an outer membrane segment configured to move relative to an inner membrane segment around a hinge linking the outer membrane segment to the inner membrane segment. The outer membrane segment has a radially outward-facing surface that is non-vertical from an anterior-to-posterior direction. The lens device has an optical liquid present within the internal chamber and the volume of the deformable region; and a force translation arm extending radially outward from the deformable region. Upon implantation of the lens device in the eye, the force translation arm directly contacts ciliary tissue to harness ciliary body movements that compresses and reduces the volume of the deformable region displacing optical liquid contained within the volume of the deformable region towards the internal chamber.

The outer membrane segment can have an anterior-facing surface and a posterior-facing surface. The deformable region can have an outside height (A) between the anterior-facing surface of the outer membrane segment and the posterior-facing surface of the outer membrane segment. The outside height (A) of the deformable region can be about 0.4 mm-0.7 mm. The inner membrane segment can include an inner surface on an anterior region and an inner surface on a posterior region of the inner membrane segment. The deformable region can have an inside height (E) between the inner surface on the anterior region of the inner membrane segment and the inner surface on the posterior region of the inner membrane segment. The inside height (E) of the deformable region can be about 0.4 mm-0.8 mm. Inner surfaces of the hinge can create a narrowing in the volume of the deformable region separating an outer portion of the volume from an inner portion of the volume. The narrowing in the volume can form a nozzle segment within the deformable region. The nozzle segment can have a height (B) between an inner surface of the hinge on an anterior side and an inner surface of the hinge on a posterior side. The height (B) of the nozzle segment can be about 0.15 mm-0.4 mm.

The inner membrane segment can include an external surface facing generally radially outward and the outer membrane segment can include an external surface facing generally radially inward. There can be a distance between the external surface of the inner membrane segment and the external surface of the outer membrane segment and an angle between the external surface of the inner membrane segment and the external surface of the outer membrane segment. The distance between the external surface of the inner membrane segment facing generally radially outward and the external surface of the outer membrane segment facing generally radially inward can be about 0.2 mm-0.6 mm. The distance and the angle can be configured to change upon application of a force causing movement of the outer membrane segment relative to the inner membrane segment. The non-vertical radially outward-facing surface can have an anterior region that extends further radially outward than a posterior region of the outer membrane segment. The non-vertical radially outward-facing surface can form an angle with the AIOL plane relative to the Z-axis that corresponds to an angle of motion of the force translation arm.

An anterior region of the outer membrane segment can compress a first distance upon application of a first force to the anterior region and a posterior region of the outer membrane segment can compress a second distance upon application of a second force. The first distance and the second distance can be the same and the first force and the second force can be different. The force translation arm can include a sloped external surface where direct contact between the force translation arm and ciliary tissue occurs during ciliary body movements.

### BRIEF DESCRIPTION OF THE DRAWINGS

These and other aspects will now be described in detail with reference to the following drawings. Generally speaking, the figures are not to scale in absolute terms or comparatively but are intended to be illustrative. Also, relative placement of features and elements may be modified for the purpose of illustrative clarity.

FIG. 1A is a perspective view of an intraocular lens;

FIG. 1B is a side view of the intraocular lens of FIG. 1A;

FIG. 1C is a cross-sectional view of the lens of FIG. 1B taken along line A-A;

FIG. 2 is a cross-sectional view of an AIOL;

FIG. 3A illustrates a top view of an accommodating intraocular lens device;

FIG. 3B illustrates a cross-sectional, partial view of the device of FIG. 3A taken along section A-A;

FIG. 3C illustrates another implementation of a cross-sectional, partial view of the device of FIG. 3A taken along section A-A;

FIG. 4A is a schematic of a membrane segment in an uncompressed state;

FIG. 4B is a schematic of the membrane segment of FIG. 4A in an accommodated state;

FIG. 5A is a cross-sectional schematic of a deformable region having an angular bellows geometry having a hinge;

FIG. 5B is a cross-sectional schematic of a deformable region having a curved bellows geometry having a hinge;

FIG. 5C is a cross-sectional schematic of a deformable region having an angular bellows geometry with more than a single hinge;

FIG. 5D is a cross-sectional schematic of a deformable region having an angular bellows geometry showing a force applied at an angle;

FIG. 6 shows a deformable region having a curved bellows geometry illustrating various parameters tested;

FIG. 7A is a vertical membrane segment shown in cross-section;

FIG. 7B is a curved bellows deformable region formed by inner and outer membrane segments shown in cross-section;

FIG. 7C shows displaced volume of the vertical membrane segment of FIG. 7A compared to the curved bellows deformable region of FIG. 7B upon application of a force by a force translation arm.

It should be appreciated that the drawings herein are exemplary only and are not meant to be to scale.

### DETAILED DESCRIPTION

The present disclosure relates generally to the field of ophthalmics, more particularly to ophthalmic devices, including intraocular lenses (IOLs) such as accommodating intraocular lenses (AIOLs). The dynamic nature of AIOLs allows for a large, continuous range of focusing power, just as in a young accommodative natural eye. The devices described herein can provide focusing power across the full accommodative range from distance to near by mechanically and functionally interacting with eye tissues typically used by a natural lens such as the ciliary body, ciliary processes, and the zonules, to effect accommodation and disaccommodation. The forces generated by these tissues are functionally translated to the devices described herein causing a power change to more effectively accommodate. The devices described herein are configured to be adjusted for size and fit prior to, during, as well as at any time after implantation. The devices described herein can be implanted in the eye to replace a diseased, natural lens. It should be appreciated, however, the devices can also be implanted as a supplement of a natural lens (phakic patient) or an intraocular lens previously implanted within a patient's capsular bag (pseudophakic patient).

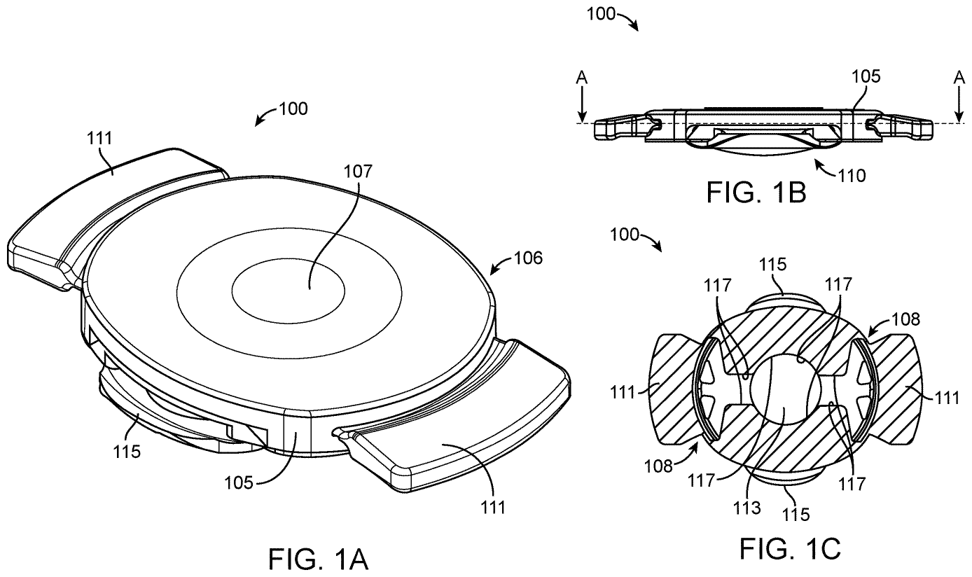

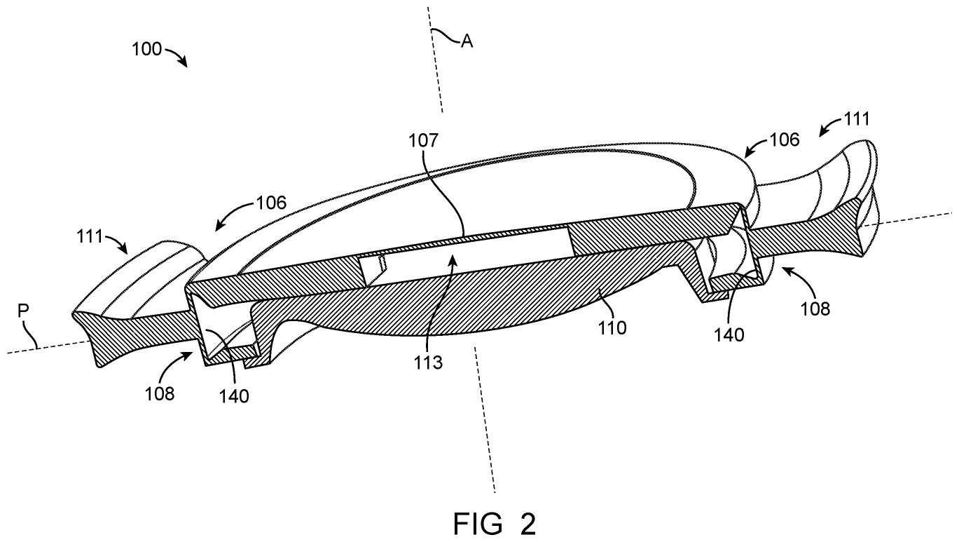

FIGS. 1A-1C illustrate an implementation of an accommodating intraocular lens 100 including an anterior lens capsule 105, a posterior lens structure 110, and a haptic 115. FIG. 1C shows a cross-sectional view of the lens 100 revealing an internal chamber 113. The internal chamber 113 is bound, in part, by surfaces 117 located between the anterior capsule 105 and posterior lens structure 110 and is configured to hold a volume of liquid such as silicone oil (e.g., silicone or fluorosilicone oil). The anterior lens capsule 105 can include a dynamic anterior optic 107 that can change shape for purposes of accommodation.

One, preferably two, force translation arms 111 can extend radially outward from a perimeter region 106 of the lens capsule 105 that are configured to drive shape change of the anterior optic 107. A deformable region 108 can be defined by at least one membrane segment 140 extending along an arc length of the perimeter region 106 (see FIG. 2). As will be described in more detail below, upon implantation of the lens device in the eye, movements of the ciliary body are harnessed directly by the force translation arms 111 to cause a change in the geometry of the deformable region 108 (e.g., movements of the membrane segment 140) thereby deforming the optical fluid in the sealed internal chamber 113 to cause a change in the shape of the dynamic anterior optic 107 of the lens capsule 105.

Again, with respect to FIG. 2, the membrane segment 140 can extend along an arc length of the perimeter region 106. The arc length can be sufficient, either individually or in combination with other membrane segments 140, to cause a reactive shape change in the dynamic anterior optic 107 upon inward (or outward) movement of the membrane segment 140. Movement of the membrane segment 140 in a generally inward direction towards the optical axis A of the AIOL 100 during accommodation can cause outward flexure or bowing of the dynamic anterior optic 107 without affecting the overall optic zone diameter in any axis. The membrane segment 140 can have a flexibility such that it is moveable and can undergo displacement relative to the lens capsule 105. For example, the membrane segment 140 can be more flexible than adjacent regions of the lens such that it is selectively moveable. The membrane segment 140 can have a resting position. The resting position of the membrane segment 140 can vary. In some implementations, the resting position is when the membrane segment 140 is positioned generally perpendicular to a plane P parallel to the anterior optic 107 such that it has a cross-sectional profile that is vertically oriented, parallel to the optical axis A.

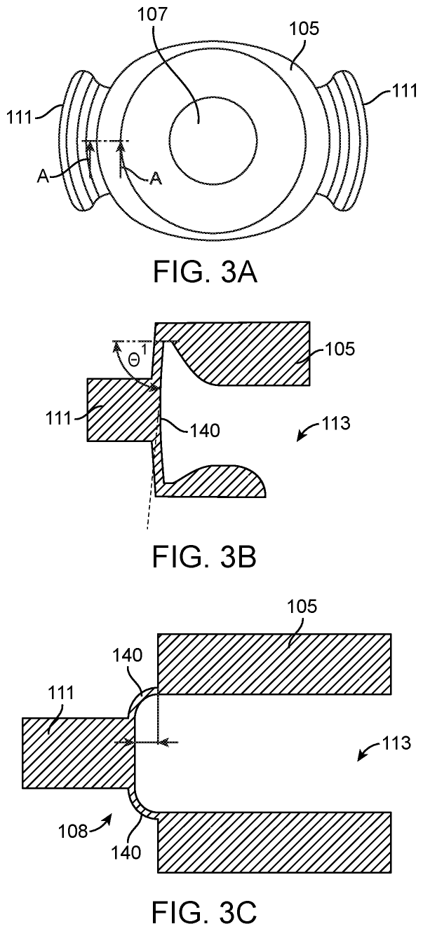

The resting position of the membrane segment 140 can also be angled relative to the optical axis A of the lens capsule 105. As shown in FIGS. 3A-3B, the cross-section of the membrane segment 140 may be angled peripherally at an angle Θ¹ relative to the lens capsule 105. In some implementations, the angle Θ¹ is between 45-89 degrees. In some implementations, the Θ¹ is 80-89 degrees. Alternatively, the cross-sectional profile of the membrane segment 140 may be a curvilinear structure protruding peripherally from the optical axis A of the lens capsule 105 thereby defining a deformable region 108 projecting radially outward from the perimeter region 106 of the lens capsule 105 (see FIG. 3C). The peripheral protruding membrane segments 140 may protrude peripherally 0.05 mm-0.5 mm. In some implementations, the curvilinear protrusion extends 0.1 mm-0.3 mm away from optical axis A of the lens capsule 105 relative to the equator region. The shape and relative arrangement of the one or more membrane segments 140 provides the lens with a low force, low movement, high accommodative function, as will be described in more detail below.

The type of movement of the membrane segment 140 is dependent upon the geometry of the deformable region 108. Preferably, the deformable region 108 can have a geometry that is configured to undergo compression, collapse, deflection, displacement, hinging or other type of mechanical movement of the membrane segments 140 relative to one another. This geometry of the deformable region 108 avoids stretching or elongating the membrane segments 140 as this sort of deformation requires higher forces to achieve accommodation compared to the force needed to achieve bending or hinging of membrane segments relative to one another.

The deformation can be in a first direction (such as generally toward an optical axis A of the lens capsule 105) upon application of a force on the deformable region 108, such as by the force translation arms 111. The movement of the membrane segments 140 of the deformable region 108 can be located inside or, preferably, outside the optic zone. Upon release of the force on the deformable region 108, the membrane segments 140 and/or other components of the AIOL 100 (e.g., the optical fluid filling the sealed internal chamber 113) can have elastic memory such that the membrane segments 140 return towards their resting position. Depending on the coupling of the AIOL 100 within the eye, the membrane segments 140 can also be pulled outward away from the optical axis A of the AIOL 100.

The membrane segments 140 lie adjacent or are coupled to or integrated with a respective force translation arm 111. In some implementations, the force translation arm 111 is moved inwardly toward the optical axis A of the AIOL 100 due to ciliary muscle contraction and applies a force against the membrane segments 140. Upon ciliary muscle relaxation, the membrane segments 140 return to their resting position and the force translation arm 111 returns to its resting position. The elastomeric nature of the movable components (i.e., the dynamic anterior optic and/or the membrane segments) can cause a return of the force translation arms 111 to their resting position. Upon ciliary muscle contraction, the force translation arm 111 and membrane segments 140 move in concert from a resting position to a generally inwardly-displaced position causing shape change of the dynamic anterior optic 107.

The number and arc length of each deformable region 108 around a perimeter of the lens capsule 105 can vary and can depend on the overall diameter and thickness of the device, the internal volume, refractive index of the material, etc. Depending on the overall diameter and thickness of the AIOL 100, the arc length around a perimeter of the lens capsule 105 of the deformable region 108 can be at least about 2 mm to about 8 mm. In some implementations, the AIOL has a single deformable region 108 formed of one or more membrane segments 140 with an arc length around a perimeter of the lens capsule 105 of between about 2 mm to about 8 mm. The single deformable region 108 can be designed to move between about 10 μm and about 100 μm upon application of forces as low as about 0.1 grams of force (gf) to achieve at least a 1D, or 1.5D, or 2D, or 2.5D, or 3D change in the dynamic anterior optic 107. In another implementation, the AIOL can have two, opposing deformable regions 108 each having an arc length around the perimeter of the lens capsule 105 that is between about 3 mm and about 5 mm. The deformable region 108 can be designed to move between about 25 μm and about 100 μm each upon application of about 0.25 g force to 1.0 g force achieve at least a 1D change in the dynamic anterior optic 107.

The membrane segments 140 of the deformable region 108 can move or collapse relative to the rest of the lens body upon application of a degree of force. Generally, the AIOL is designed such that very low forces are sufficient to cause micron movements to cause sufficient diopter changes and with reliable optics. The force applied to achieve movement of the dynamic anterior optic 107 of the lens body 105 to effect accommodation can be as low as about 0.1 grams of force (gf). In some implementations, the force applied can be between about 0.1 gf to about 5.0 gf or between about 0.25 gf to about 1.0 gf or between about 1.0 gf to about 1.5 gf. The movements of the deformable regions 108 of the lens body 105 (e.g., membrane segment 140) relative to the central portion of the lens body 105 (e.g., dynamic anterior optic 107) in response to forces applied to achieve accommodation can be as small as about 50 μm. The movements of the deformable region 108 of the lens body 105 relative to the dynamic anterior optic 107 in response to forces applied can be between about 50 μm to about 500 μm, between about 50 μm to about 100 μm, between about 50 μm to about 150 μm, or between about 100 μm to about 150 μm. The ranges of forces applied (e.g., about 0.1 gf to about 1 gf) that result in these ranges of movement in the deformable region 108 (e.g., 50 μm-100 μm) can provide the devices described herein with an accommodating capability that is within a dynamic range of greater than at least ±1D and preferably about ±3 diopters (D). In some implementations, the power is about ±1D for about 20 μm movement, or about ±2-3D for about 40 μm movement, or about ±4-5D for about 60-80 μm movement. The devices described herein can have an accommodating range that is at least ±1D for about 20 μm movement of the deformable region 108 and about a force of at least 0.25 gf applied to the deformable region 108. In other implementations, the devices can have an accommodating range that is at least ±4D for about 40-80 μm movement and at least about 1.0 gf. In other implementations, the devices can have an accommodating range that is at least ±4D for about 50 μm movement and at least about 0.5 gf. The available movement is preferably in a range of 40-80 microns in response to an available force in a range of 1-1.5 gf. Upon release of the force, the deformable region 108 preferably returns to original shape within a short period of time that is less than 0.1 seconds, preferably less than 0.8 seconds, more preferably about 0.2-0.6 seconds.

The optical fluid filling the sealed internal chamber 113 can be a non-compressible optical fluid and the volume of the sealed chamber 113 can be substantially identical to the volume of optical fluid. As such, the optical fluid filling the chamber 113 does not cause significant outward bowing of either the dynamic anterior optic 107 or the deformable region 108 in the resting state when no substantial outside forces are applied to the AIOL 100. In some implementations, the internal chamber 113 can be slightly overfilled with optical fluid such that the dynamic anterior optic 107 has some outward bowing at rest. A small degree of resting outward bowing in the dynamic anterior optic 107 can reduce optical artifacts in the lens. However, no matter how much resting outward bowing is present in the dynamic anterior optic 107, the anterior optic 107 can still undergo additional outward bowing upon application of compressive forces on the deformable region 108 to provide accommodation. The pressure inside the sealed chamber 113 can be substantially equal to the pressure outside the sealed chamber 113. Because the optical fluid in the sealed chamber 113 is non-compressible its shape deforms along with the shape of the chamber 113. Deformation of the chamber 113 in one location (e.g., micrometer inward movements of the outer membrane segment 140b) causes the non-compressible optical fluid present within the fixed-volume sealed chamber 113 to press against the inner-facing surfaces 117 forming the sealed chamber 113. A reactive deformation of the sealed chamber 113 occurs in a second location to create sufficient accommodating change. The dynamic anterior optic 107 is configured to bow outward upon application of a force (e.g., due to relative thickness and/or elasticity) compared to other parts of the anterior optic 107 such as a perimeter region 106. Thus, inward movement of deformable region 108 urges the optical fluid to deform along with the chamber 113 and press against the inner-facing surface of the anterior optic 107. This results in outward bowing and reshaping of the outer surface of the dynamic anterior optic 107 to cause the accommodative portion of the optic zone to become more convex increasing the power of the AIOL 100.

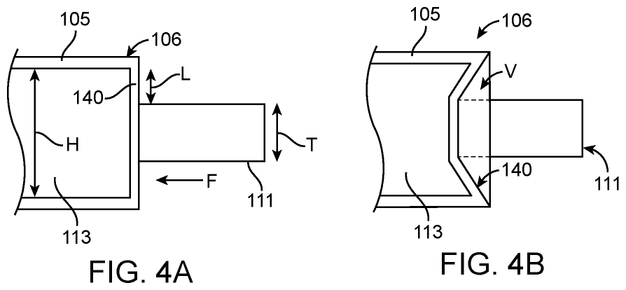

Again, with respect to FIG. 2, a membrane segment 140 is shown schematically arranged substantially vertical or aligned parallel with the optical axis. Vertical membrane segments 140, like the one shown in FIG. 2, elongate and stretch when placed under compression. FIG. 4A illustrates a vertical membrane segment 140 of a lens in schematic in an uncompressed state. The force translation arm 111 can be urged inward by a force F, such as upon ciliary body contraction, and translate that motion against the membrane segment 140 (see FIG. 4B). The membrane segment 140 upon motion of the force translation arm 111 to the accommodated state elongates or stretches to move inward and displace a volume V of optical liquid inside the deformable region 108 of the lens device 100. Membrane elongation requires higher forces be applied to achieve displacement of a fluid volume. The membrane segment(s) 140 are preferably arranged so that they bend or hinge relative to one another, rather than stretch or elongate, to achieve displacement of optical material within the lens device with the relatively low available force. The bending or hinging of the membrane segment(s) 140 increases the displaced volume of optical liquid at lower applied forces as discussed in more detail below.

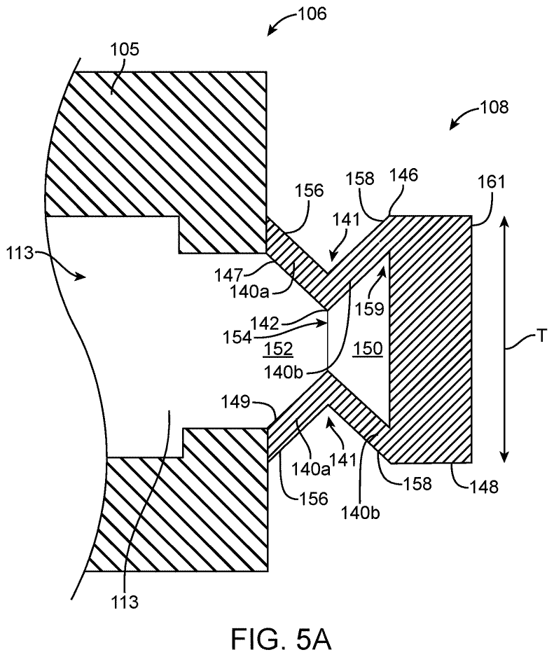

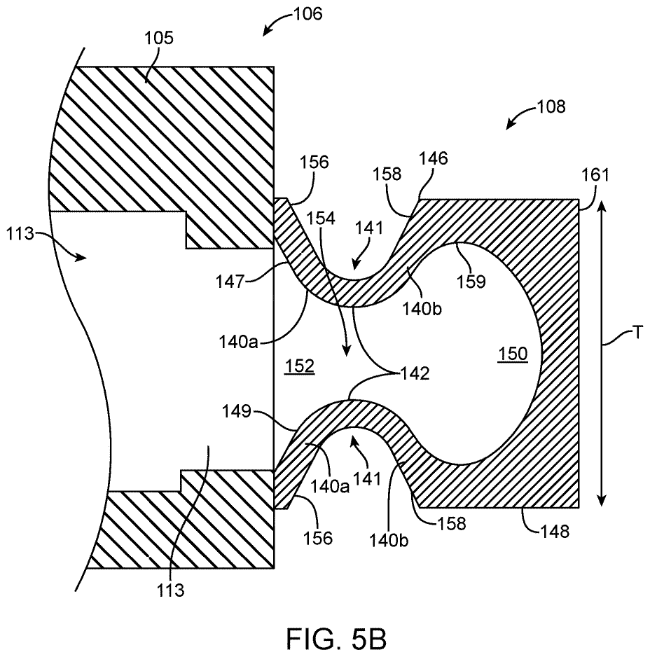

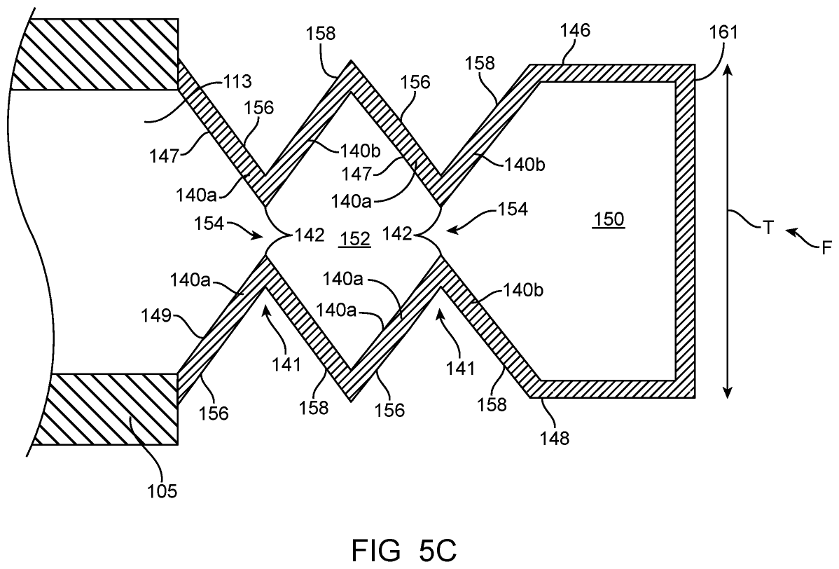

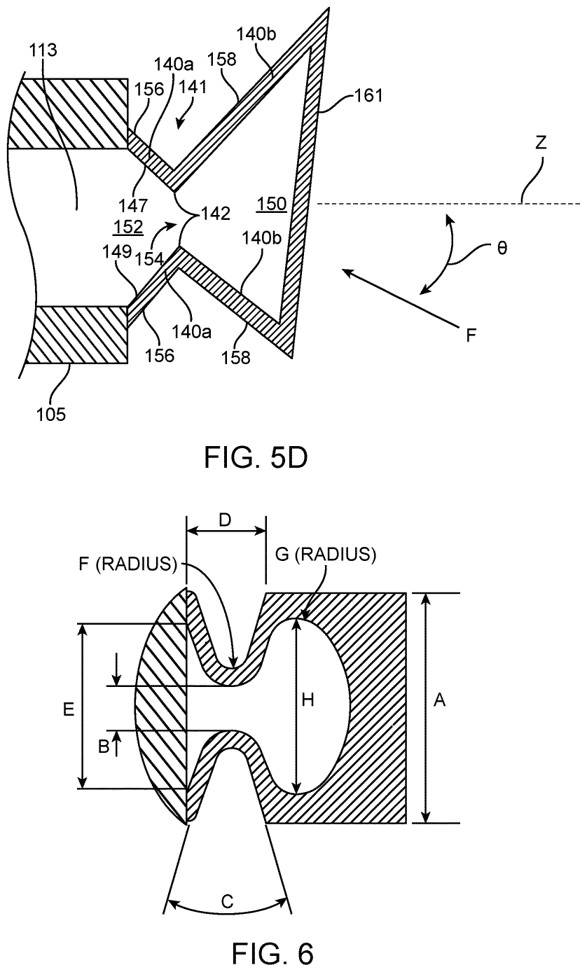

FIGS. 5A-5D illustrate in schematic implementations of a lens device having a lens capsule 105 having an internal chamber 113 bound, in part, by a dynamic anterior optic (not visible in FIGS. 5A-5D) and a deformable region 108 defined by membrane segments 140a, 140b that project radially outward from the perimeter region 106 of the lens capsule 105. The deformable region 108 has a three-dimensional shape defining an internal volume. The optical liquid of the lens device is present within both the internal chamber 113 of the lens capsule 105 and the volume of the deformable region 108. A force translation arm (not visible in FIGS. 5A-5D) can extend radially outward from the deformable region 108. Upon implantation of the lens device in the eye, the force translation arm 111 harnesses directly (i.e., by being in direct contact with the tissue) ciliary body movements, which are generally in the direction of arrow F shown in FIGS. 5C-5D, to compress and thereby reduce the volume of the deformable region 108 displacing optical liquid present within the volume of the deformable region 108 towards the internal chamber 113. The deformable region 108 projecting radially outward from the perimeter region 106 of the lens capsule 105 is formed by the membrane segments 140a, 140b that are configured to temporarily bend or collapse relative to one another around at least one hinge 141 during accommodation to achieve displacement of optical material inside of the lens device. The hinging of the membrane segments 140a, 140b is achieved due to the geometry of the deformable region 108 formed by the segments 140a, 140b being, for example, in the shape of a bellows.

The deformable region 108 can have various three-dimensional geometries that are formed by the membrane segment(s) 140 that bend relative to one another upon application of a force against the deformable region 108 by the force translation arm 111. FIG. 5A shows an angular bellows shape and FIG. 5B shows a curved bellows shape. Each of the figures shows the chamber 113 internal to the deformable region 108 and located between an anterior and a posterior segment of the lens capsule 105. A volume is defined by the three-dimensional shape of the deformable region 108. The bellows shaped deformable region 108, when compressed, acts as a positive displacement pump to urge optical liquid from within the volume of the deformable region 108 towards the internal chamber 113 of the lens capsule 105 for accommodation. The bellows geometry encourages collapse of membrane segments 140a, 140b around the hinge 141 and internal expansion within a region of the lens that causes outward bowing of the anterior optic 107. The bellows geometry ensures that even with relatively thin, elastomeric membrane segments 140a, 140b, the expansion occurs within the lens as opposed to an inadvertent outward expansion within the external deformable region itself. The angular bellows configuration of FIG. 5A (and also FIGS. 5C and 5D) has anterior and posterior portions that each include an inner membrane segment 140a that projects away from the perimeter region 106 of the lens capsule 105 at an angle and an outer membrane segment 140b that projects out from the inner membrane segment 140a. The membrane segments 140a, 140b of the angular bellows deformable region 108 in this configuration can collapse or bend at hinge 141 such that their outer surfaces fold or bend towards one another thereby decreasing the volume of the deformable region 108 due to inward motion of the force translation arm 111. The optical liquid is displaced from within the volume of the deformable region 108 towards the internal chamber 113 of the lens capsule 105 to cause shape change of the anterior optic 107. The curved bellows deformable region 108 of FIG. 5B also has anterior and posterior portions each including an inner membrane segment 140a and an outer membrane segment 140b. The membrane segments 140a, 140b of the curved bellows deformable region 108 collapse such that their outer surfaces fold towards one another to decrease the volume of the deformable region due to inward motion of the force translation arm 111. FIG. 5C illustrates a deformable region 108 formed by a plurality of membrane segments movable around more than one hinge 141 creating a plurality of bellows. The bellows designs that bend around the hinge are capable of capturing and translating force of the ciliary muscle movements more efficiently. The multiple bellows can provide the same amount of compression with even less force compared to a bellows with a single hinge. The displacement mechanism of the deformable region 108 can be compared to a spring. The forces required to displace the spring can be calculated using Hook's low formula F=k*x, where (k) is the spring constant, (F) denotes the force, and (x) denotes the change in spring compression/elongation (x). Spring constant (k) in relation to the number of spring active coils (na) is inversely proportional (based on the Formula for calculation of the spring constant: k=Gd\hat{ }4/[8\hat{ }3D*na], where (G) is shear modulus of material, (d) denotes spring wire diameter, (D) denotes outer diameter of spring, (na) denotes number of active coils. Increasing the number of bellows (hinges) of the deformable region 108 decreases the spring constant (k). If the spring constant (k) is smaller, the force (F) needed to displace the spring at the same distance (x) is also smaller.

The motion of the force translation arm 111 can be directly inward parallel to a Z-axis or may be tilted (e.g., 5-10 degree angle θ from the AIOL plane) relative to the Z-axis (see FIG. 5D). The tilt or angle that the force translation arm 111 projects posteriorly is selected to match the angle the ciliary body moves anteriorly. During contraction, movement of the ciliary muscle occurs in two planes—radially inward and also anteriorly (i.e., toward the corneal plane) by about 5-10 degrees. This angled design of the force translation arm 111 relative to the plane of the AIOL captures movement of the ciliary body and translates the force more efficiently into displacement of optical liquid from the deformable region 108 toward the center of the lens body so that the dynamic anterior optic bows for accommodation. Additionally, the bellows designs that bend around a hinge (whether a single hinge or multiple hinges) capture and translate ciliary movements that are not parallel to the plane of the AIOL. FIG. 5D shows a force F being applied such as by force translation arms 111 that project posteriorly at an angle that corresponds to the angle of anterior ciliary body movement. The radially outward-facing surface 161 of the bellows region is non-vertical from an anterior-to-posterior direction, such that an anterior region of the outer membrane segment 140b extends further radially outward than a posterior region of the outer membrane segment 140b. The angle formed by the non-vertical radially outward-facing surface 161 is intended to accommodate the motion of the force translation arm 111 directly inward at an angle θ from the AIOL plane relative to the Z-axis (e.g., 5-10 degrees). Also, the bellows design incorporating a hinge aids in translating that movement coming from an angle.

The force translation arms 111 can alternatively or additionally incorporate a sloped external surface where contact with the ciliary tissue occurs. The slope where contact is made with the ciliary processes “absorbs” and transfers the force more efficiently. With a membrane 140 as shown in FIGS. 4A-4B that is vertical, there is a risk that membrane movement above and below where the force translation arms 111 contact the membrane 140 may be unequal with a sloped force translation arm 111. If the portion of the membrane 140 above the arm 111 moves inward prior to the portion of the membrane 140 below the arm 111, the liquid within the deformable region 108 may be displaced from the portion below the arm 111 to the portion above the arm 111, which negatively impacts the efficiency of shape change within the optical portion of the lens.

The force transfer arm and/or bellows can be designed to undergo angled movement to better capture the corresponding angled movements of the ciliary body thereby maximizing the movement of the deformable region of the lens. In a first approach, the angle of ciliary movement can be integrated into the AIOL periphery, such as by designing the bellows at an angle corresponding to the angled movement of the ciliary body as described above with regard to FIG. 5D. Alternatively, the external surface of the force transfer arms can be designed to have a slope as described in more detail below. In a second approach, the bellows can be designed asymmetrically so that the force needed to compress one region of the membrane segment is unequal to the force needed to compress another region of the membrane segment. The posterior region of the membrane segments 140a, 140b can compress an amount (e.g., a compressible distance) upon application of a force and the anterior region of the membrane segments 140a, 140b can compress that same amount (e.g., the same compressible distance) upon application of a force that is different from the force needed to compress the posterior region. For example, the force needed to compress the posterior region can be less than the force needed to compress the anterior region of the membrane segments that same amount or vice versa.

Where the deformable region 108 is formed by a membrane segment 140 that is vertical and has a length L lying perpendicular to the force direction (arrow F in FIG. 4A), the membrane 140 must stretch and elongate when compressed by the rigid force translation arm 111. The thickness T of the force translation arm 111 compressing the membrane 140 is limited by the flexible membrane 140 length L and must be smaller than the total height H of the internal chamber 113 (see FIG. 4A). In contrast, a deformable region 108 formed by membrane segments 140a, 140b arranged in a bellows geometry and movable around at least one hinge 141 can having a force translation arm 111 thickness T that is sized smaller than, the same as, or larger than a total height H of the internal chamber 113 (see FIGS. 5A-5C). If a force is applied by a force translation arm 111 against a vertical membrane segment 140 like that shown in FIG. 4A and the arm 111 is at an angle, the uneven movement of the membrane 140 can cause one membrane segment to stretch into the lens while the other membrane segment stretches at an opposite direction resulting in less displacement of optical liquid and unpredictable outward bowing and accommodation. In the bellows geometry, the force F can be applied at an angle θ and still achieve efficient displacement of optical liquid within the deformable region 108 and predictable accommodation of the lens.

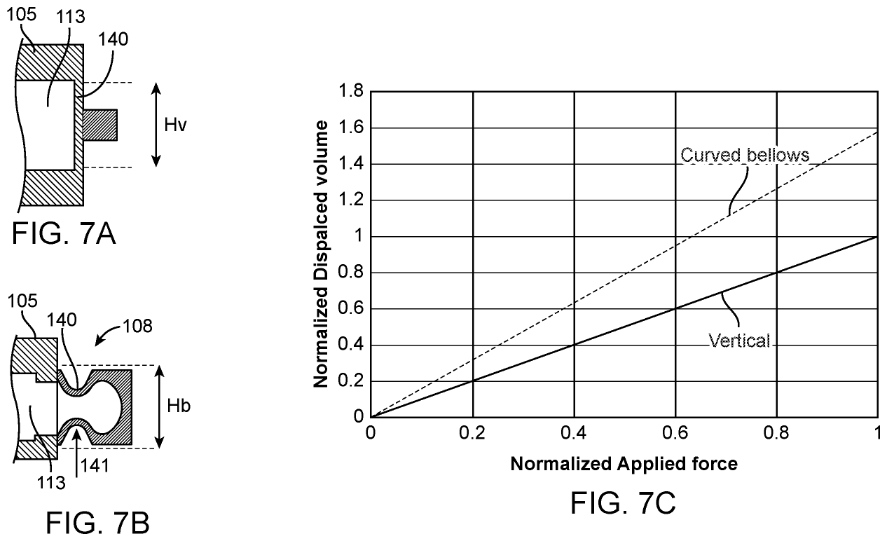

Again, with respect to FIG. 5A-5B and also FIG. 7B, the bellows-shaped deformable region 108 can have an inner membrane segment 140a linked to an outer membrane segment 140b by a hinge 141, wherein the outer membrane segment 140b is configured to move relative to the inner membrane segment 140a around the hinge 141. The outer membrane segment 140b includes an anterior-facing surface 146 and a posterior-facing surface 148. Correspondingly, the inner membrane segment 140a includes an inner surface 147 on an anterior region near the internal chamber 113 and an inner surface 149 on a posterior region of the inner membrane segment 140a near the internal chamber 113. The inner surfaces 142 of the hinge 141 creates a narrowing in the volume of the deformable region 108 separating an outer portion 150 of the volume from an inner portion 152 of the volume of the deformable region 108. The narrowing forms a nozzle segment 154 within the volume of the deformable region 108. FIG. 6 shows a deformable region 108 having the curved bellows geometry illustrating various parameters including (A) outside height of the outer membrane segment 140b from the anterior-facing surface 146 of the outer membrane segment 140b to the posterior-facing surface 148 of the outer membrane segment 140b, (B) height of the nozzle segment 154 between an inner surface 142 of the hinge 141 on an anterior side and an inner surface 142 of the hinge 141 on a posterior side, (C) angle between an external surface 156 the inner membrane segment 140a facing generally radially outward and an external surface 158 of the outer membrane segment 140b facing generally radially inward, (D) collapsible distance between the radially outward external surface 156 of the inner membrane segment 140a and the radially inward external surface 158 of the outer membrane segment 140b, (E) inside height of inner membrane segment 140a between the inner surface 147 on the anterior region of the inner membrane segment 140a near the internal chamber 113 and the inner surface 149 on the posterior region of the inner membrane segment 140a near the internal chamber 113, (F) radius of the inner surface 142 of the hinge 141 at the nozzle segment 154, (G) radius of the inner surface 159 of the outer membrane segment 140b forming an anterior region of the outer portion 150 of the volume of the deformable region 108. The internal height (H) of the internal chamber 113 is shown in FIG. 6. The radius (G) and internal height (H) are parameters that describe how easily the segments 140b can deform. If radius (G) of the inner surface 159 of the outer membrane segment 140b and the internal height (H) of the internal chamber 113 are too large, then the posterior-facing surface 148 and/or anterior-facing surface 146 will deform too easily with ciliary movement, reducing the total oil volume effectively displaced to the dynamic optic. If radius (G) of the inner surface 159 of the outer membrane segment 140b and/or the internal height (H) of the internal chamber 113 are kept small, the outer chamber will be more rigid and all the deformation with ciliary movement will be concentrated in inner membrane segment 140a and outer membrane segment 140b, which provides a more effective displacement of the total oil volume to the dynamic optic. However, if the internal height (H) of the internal chamber 113 are too small then the membrane segments 140a, 140b are shortened thereby reducing their ability to suitably deform. Table 1 below lists relevant ranges for each of these parameters.

TABLE 1

| Parameter | Relevant range |
|---|---|
| A | 0.4-0.7 mm |
| B | 0.15-0.4 mm |
| C | 30°-120° |
| D | 0.2-0.6 mm |
| E | 0.4-0.8 mm |
| F | Corner or radius between 0.13-0.2 mm |
| G | Corner or radius between 0.05-0.2 mm |
| H | 0.3-0.6 mm |

Simulations were performed to compare the performance of a vertical membrane segment 140 (FIG. 7A) to a curved bellows deformable region 108 having an inner membrane segment 140a linked to an outer membrane segment 140b by a hinge (FIG. 7B). The vertical membrane segment 140 of FIG. 7A has a height Hv in an anterior-to-posterior direction that is greater than a corresponding height of the curved bellows deformable region 108 between the anterior-facing surface 146 of the outer membrane segment 140b and the posterior-facing surface 148 of the outer membrane segment 140b. For example, the anterior-to-posterior height Hv of the deformable region 108 in the vertical membrane embodiment of FIG. 7A can be about 0.70 mm to about 0.75 mm and the corresponding anterior-to-posterior height Hv of the deformable region 108 in the curved bellows embodiment of FIG. 7B can be about 0.60-0.65 mm. In some implementations, the curved bellows deformable region can have an overall height Hb in the anterior-to-posterior direction (i.e., the outside height between the anterior-facing surface 146 and posterior-facing surface 148 of the outer membrane segment 140b) that is about 0.5 mm (i.e., 0.45 mm-0.55 mm). FIG. 7C shows the curved bellows deformable region 108 of FIG. 7B provided 60% more displaced volume compared to the vertical membrane segment of FIG. 7A at a given applied force despite the overall shorter height.

Table 2 below illustrates examples of displaced volumes provided by deformable regions having different geometries, the vertical membrane embodiment, similar to what is shown in FIG. 7A, can have an inside height E of the deformable region, or the height of inner membrane segment 140a between the inner surface 147 on the anterior region of the inner membrane segment 140a and the inner surface 149 on the posterior region of the inner membrane segment 140a near the internal chamber 113, that is about 0.72 mm. The displaced volume is about 0.1 mm³-0.2 mm³ (or about 0.15 mm³) upon 60 μm ciliary movement and applying about 1.4-2.0 gf. The curved bellows embodiment, similar to what is shown in FIG. 7B, can have an inside height E that is about 0.63 mm, a displaced volume is about 0.2 mm³-0.5 mm³ (or about 0.3 mm³) upon 60 μm ciliary movement and applying about 1.3-1.7 gf. The displaced volume of the curved bellows embodiment having an inside height E about 0.45 mm-0.55 mm (preferably about 0.5 mm) and is about 0.15 mm³-0.35 mm³ (preferably about 0.2 mm³) upon 60 μm ciliary movement and applying about 1.3-2.0 gf. The material displacement capability of the curved bellows deformable region 108 is relatively high without increasing the force required to activate the force translation arms and provides this capability with an overall narrower lens.

TABLE 2

| | Anterior-to- | Applied force at | |
|---|---|---|---|
| | posterior height | 60 μm ciliary | |
| | of deformable | movement | Displaced volume |
| Geometry | region (mm) | (grams force) | (mm³) |
| Vertical | 0.72 | 1.4-2.0 | 0.1-0.2 |
| Curved Bellows | 0.63 | 1.3-1.7 | 0.2-0.5 |
| Curved Bellows | 0.50 | 1.3-2.0 | 0.15-0.35 |

The bellows deformable region 108, whether curved or angled at the hinge 141, can achieve sufficient accommodation with a very small displacement of the outer membrane segment 140b in a range of about 50-60 microns movement. In the curved bellows configuration, application of a force F against a radially outward-facing surface 161 of the outer membrane segment 140b decreases the collapsible distance D between the external surface of the inner membrane segment facing generally radially outward and the external surface of the outer membrane segment facing generally radially inward. This also decreases the radius F of the deformable region at the nozzle segment and narrows the angle C between the external surface 156 of the inner membrane segment 140a facing generally radially outward and the external surface 158 of the outer membrane segment 140b facing generally radially inward. The relative movement of the inner membrane segment 140a and the outer membrane segment 140b upon movement of the force transfer arms is dependent upon the specific geometry. Where the outer membrane segment 140b is configured to move along with the motion of the force translation arm as the force is applied, the inner membrane segment 140a may be configured to move only minimally with the motion of the force translation arm. In some implementations, the inner membrane segment 140a moves with movement of the force transfer arms and the outer membrane segment 140b does not move. In other implementations, the inner membrane segment 140a and the outer membrane segment 140b move equally with movement of the force transfer arms. The curvature of the inner membrane segment 140a relative to the lens body can undergo a slight flattening inwards toward the internal chamber 113.

The lens 100 can be formed of a material configured for small incision implantation. The solid optical components of the lens are substantially elastomeric and can be made of soft silicone polymers that are optically clear, biocompatible, and in certain circumstances flexible having a sufficiently low Young's modulus to allow for the lens body to change its degree of curvature during accommodation. Suitable materials for the solid optical component of the lens can include, but are not limited to silicone (e.g., alkyl siloxanes, phenyl siloxanes, fluorinated siloxanes, combinations/copolymers thereof), acrylic (e.g., alkyl acrylates, fluoroacrylates, phenyl acrylate, combinations/copolymers thereof), urethanes, elastomers, plastics, combinations thereof, etc. In aspects, the solid optical component of the lens is formed of a silicone elastomer, as described herein. The solid optical component can be formed of one or a combination of the materials described herein in which the liquid optical material described herein is fully encapsulated by the solid optical component. The solid optical component of a lens may include one or more regions that are configured to be in contact with and/or contain the liquid optical material. The liquid optical materials described herein can be specially formulated relative to the material of the solid optical component to mitigate lens instability and optimize optical quality. The liquid optical materials, sometimes referred to herein as an optical fluid, can include any of a variety of copolymers, including fluorosilicone copolymers and other liquid optical materials as described in PCT Application No. PCT/US2021/37354, filed Jun. 15, 2021, which is incorporated by reference herein in its entirety.

The hardness (durometer) of the membrane segments 140 can be as low as about 10 Shore A up to about 60 Shore A. The membrane segments 140 can have a tensile strength between 600-1400 psi, an elongation that is between 150-1100%, and a tear strength that is between 30-250 ppi. The membrane segments 140 can be optically clear, translucent, or opaque as they are generally outside the visual field of the lens. The force translation arms 111 can have a high elongation and high tear force and can be between 30-60 Shore A durometer (e.g., MED-6233, MED-4244, MED 5/4830, MED 5/4840, MED 5/4850) to achieve material displacement function. The force translation arms 111 are at the perimeter of the lens and outside the visual zone and need not be optically clear. The haptic 115, like the force translation arms 111, need not be optically clear and is preferably opaque or translucent with a white colored pigment. The haptic 115 can be between 50-80 Shore A durometer (e.g., MED-5/4880, MED-5/4870, MED-5/4860, MED-5/4850). The dynamic anterior optic 107, in contrast, must be optically clear. The posterior lens structure 110 must also be optically clear and preferably has a high refractive index (>1.43). The posterior lens structure 110 can be between 30-70, preferably about 30-50 Shore A durometer (e.g., MED-6820, MED1-6755) whereas the anterior optic 107 can be between 30-50 Shore A durometer (e.g., MED1-6755, MED-6233, MED-6820).

The terms “anterior” and “posterior” as used herein are used to denote a relative frame of reference, position, direction or orientation for understanding and clarity. Use of the terms is not intended to be limiting to the structure and/or implantation of the lens. For example, the orientation of the lens capsule 105 within the eye can vary such that the anterior optic 107 can be positioned anteriorly along the optical axis A of the AIOL 100 and the posterior lens structure 110 positioned posteriorly along the optical axis A of the AIOL 100 relative to the eye anatomy. However, the anterior optic 107 can be positioned posteriorly and the posterior lens structure 110 positioned anteriorly relative to the eye anatomy.

In aspects, description is made with reference to the figures. However, certain aspects may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detain in order to not unnecessarily obscure the description. Reference throughout this specification to “one embodiment,” “an embodiment,” “an aspect,” “one aspect,” “one implementation, “an implementation,” or the like, means that a particular feature, structure, configuration, or characteristic described is included in at least one embodiment, aspect, or implementation. Thus, the appearance of the phrase “one embodiment,” “an embodiment,” “one aspect,” “an aspect,” “one implementation, “an implementation,” or the like, in various placed throughout this specification are not necessarily referring to the same embodiment, aspect, or implementation. Furthermore, the particular features, structures, configurations, or characteristics may be combined in any suitable manner in one or more implementations.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples, embodiments, aspects, and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. A lens device for treatment of an eye, the lens device comprising:
a lens capsule comprising an internal chamber bound, in part, by a dynamic anterior optic and having a perimeter region;
a deformable region having a three-dimensional bellows shape defining a volume, the deformable region projecting radially outward from the perimeter region of the lens capsule, wherein the deformable region comprises an outer membrane segment configured to bend around a hinge relative to an inner membrane segment;
a volume of an optical liquid contained within the internal chamber and the volume of the deformable region; and
a force translation arm extending radially outward from the deformable region,
wherein, upon implantation of the lens device in the eye, the force translation arm directly contacts ciliary tissue to harness ciliary body movements that compresses and reduces the volume of the deformable region displacing optical liquid contained within the volume of the deformable region towards the internal chamber.

2. The lens device of claim 1, wherein the bellows is curved or angular.

3. The lens device of claim 1, wherein the force translation arm extends radially outward and posteriorly at an angle relative to a plane of the dynamic anterior optic.

4. The lens device of claim 3, wherein the angle is about 5 to about 10 degrees.

5. The lens device of claim 3, wherein the angle matches an angle of ciliary body movement in anterior direction relative to the lens.

6. A lens device for treatment of an eye, the lens device comprising:
a lens capsule comprising an internal chamber bound, in part, by a dynamic anterior optic, the lens capsule having a perimeter region;
a deformable region having a three-dimensional curved bellows shape defining a volume, the deformable region projecting radially outward from the perimeter region of the lens capsule, wherein the deformable region comprises an outer membrane segment configured to move relative to an inner membrane segment around a hinge linking the outer membrane segment to the inner membrane segment;
an optical liquid present within the internal chamber and the volume of the deformable region; and
a force translation arm extending radially outward from the deformable region,
wherein, upon implantation of the lens device in the eye, the force translation arm directly contacts ciliary tissue to harness ciliary body movements that compresses and reduces the volume of the deformable region displacing optical liquid contained within the volume of the deformable region towards the internal chamber.

7. (canceled)

8. The lens device of claim 6, wherein the deformable region has an outside height (A) between an anterior-facing surface of the outer membrane segment and a posterior-facing surface of the outer membrane segment.

9. (canceled)

10. The lens device of claim 6, wherein the inner membrane segment comprises an inner surface on an anterior region and an inner surface on a posterior region of the inner membrane segment.

11. The lens device of claim 10, wherein the deformable region has an inside height (E) between the inner surface on the anterior region of the inner membrane segment and the inner surface on the posterior region of the inner membrane segment.

12. (canceled)

13. The lens device of claim 6, wherein inner surfaces of the hinge creates a narrowing in the volume of the deformable region separating an outer portion of the volume from an inner portion of the volume.

14. The lens device of claim 13, wherein the narrowing in the volume forms a nozzle segment within the deformable region.

15. The lens device of claim 14, wherein the nozzle segment has a height (B) between an inner surface of the hinge on an anterior side and an inner surface of the hinge on a posterior side.

16. (canceled)

17. The lens device of claim 6, wherein the inner membrane segment comprises an external surface facing generally radially outward and the outer membrane segment comprises an external surface facing generally radially inward and a distance between the external surface of the inner membrane segment and the external surface of the outer membrane segment and an angle between the external surface of the inner membrane segment and the external surface of the outer membrane segment.

18. (canceled)

19. The lens device of claim 17, wherein the distance and the angle are configured to change upon application of a force causing movement of the outer membrane segment relative to the inner membrane segment.

20. The lens device of claim 19, wherein a curvature of the inner membrane segment relative to the lens capsule flattens.

21. The lens device of claim 6, wherein the deformable region has an inside height (E) of about 0.60 mm-0.65 mm and provides a displaced volume of optical liquid that is about 0.2 mm³-0.5 mm³ at 1.3-1.7 grams applied force.

22. (canceled)

23. The lens device of claim 6, wherein the deformable region has an inside height (E) of about 0.45 mm-0.55 mm and provides a displaced volume of optical liquid that is about 0.15 mm³-0.35 mm³ at 1.3-2.0 grams applied force.

24. (canceled)

25. The lens device of claim 6, wherein an anterior region of the outer membrane segment compresses a first distance upon application of a first force to the anterior region and a posterior region of the outer membrane segment compresses a second distance upon application of a second force, wherein the first distance and the second distance are the same and wherein the first force and the second force are different.

26. The lens device of claim 6, wherein the force translation arm comprises a sloped external surface where direct contact between the force translation arm and ciliary tissue occurs during ciliary body movements.

27. A lens device for treatment of an eye, the lens device comprising:
a lens capsule comprising an internal chamber bound, in part, by a dynamic anterior optic, the lens capsule having a perimeter region;
a deformable region having a three-dimensional angular bellows shape defining a volume, the deformable region projecting radially outward from the perimeter region of the lens capsule, wherein the deformable region comprises an outer membrane segment configured to move relative to an inner membrane segment around a hinge linking the outer membrane segment to the inner membrane segment, the outer membrane segment having a radially outward-facing surface that is non-vertical from an anterior-to-posterior direction;
an optical liquid present within the internal chamber and the volume of the deformable region; and
a force translation arm extending radially outward from the deformable region,
wherein, upon implantation of the lens device in the eye, the force translation arm directly contacts ciliary tissue to harness ciliary body movements that compresses and reduces the volume of the deformable region displacing optical liquid contained within the volume of the deformable region towards the internal chamber.

28.-44. (canceled)
